(19) United States
(12) Patent Application Publication
(10) Pub. No.: US 2015/0230590 A1
(43) Pub. Date: Aug. 20, 2015

(54) Head Worn Apparatuses and Associated Methods for Adornment or Facial Tightening Purposes

(71) Applicant: Diane Livingston, Regina (CA)

(72) Inventor: Diane Livingston, Regina (CA)

(21) Appl. No.: 14/618,301

(22) Filed: Feb. 10, 2015

Related U.S. Application Data
(60) Provisional application No. 61/940,944, filed on Feb. 18, 2014.

(51) Int. Cl. A45D 44/22; A61F 13/00

(52) U.S. Cl. CPC A45D 44/22; A61F 13/00; A61F 2013/00374

(57) ABSTRACT

A head-worn apparatus features an elongated flexible member having first and second slits therein proximate opposing ends thereof for respective engagement of first and second tufts of hair through the slits at respective locations on the user's head. Tape, clips or other securing elements are used to bundle together the strands of hair in each tuft and prevent sliding of the tufts out from the respective slits, thereby maintaining the apparatus in place in the user's hair. In one embodiment, the elongated member is connected proximately rearward of the forehead on opposite sides of the head, and is sufficiently tensioned to tighten facial areas near these locations. In another embodiment, the elongated member is used to hang one or more jewelry items in the user's hair. The flexible member has a series of holes through which additional hair can be passed to conceal it from sight beneath the user's hair.

### CROSS REFERENCE TO RELATED APPLICATIONS

This application claims benefit under 35 U.S.C. 119(e) of Provisional Application Ser. No. 61/940,944, filed Feb. 18, 2014.

### FIELD OF THE INVENTION

The present invention relates generally to head-worn apparatuses that employ a flexible member connected to one's hair at opposing sides of the head in order to adorn the user's hair with jewelry or temporarily tighten facial skin using a tensioned state of the flexible member between its connections to the wearer's hair.

### BACKGROUND OF THE INVENTION

Numerous facial tightening devices have been proposed in the prior art that make use of a strap, band or the like that is connected across the head or neck in order tighten facial areas at opposing sides of the face, including U.K. Patent GB315733 and U.S. Pat. No. 2,661,737, U.S. Pat. No. 3,524,443, U.S. Pat. No. 3,584,619, U.S. Pat. No. 3,672,363, U.S. Pat. No. 3,695,257, U.S. Pat. No. 4,239,037, U.S. Pat. No. 5,555,900, U.S. Pat. No. 5,582,585, U.S. Pat. No. 8,257,385, USRE21509. These devices include bands that attach adhesively to the skin, or attach to the hair, for example using clips.

U.S. Pat. No. 3,575,165 features a skullcap style of head-worn facelift device, in which hair is passed through a hole in the skullcap in order to secure and conceal the appliance.

Applicant has developed a new facial-tightening hair appliance that operates on the same general principle as the strap or band style devices of the prior art, i.e. wearing of a tensioned elongated member across the head in order to pull the skin taught on facial areas at opposing sides of the head, but that employs a unique solution for securing the elongated flexible member in place in this tensioned position.

The elongated member of the hair appliance can also be used as part of a head-worn apparatus for other purposes, such as to support jewelry or other adornments in the wearer's hair.

Examples of prior solutions for hanging jewelry in one's hair can be found in U.S. Pat. No. 6,581,611 and U.S. Pat. No. 6,579,11, and U.S. Patent Application Publications US20080006288, US20120103014, US20120180805, and US20120318292, none of which disclose the unique hair attachment solution employed in the present invention.

### SUMMARY OF THE INVENTION

According to a first aspect of the invention there is provided a method of applying a head-worn apparatus, the method comprising:

(a) forming a first tuft of hair on a user's head from a first plurality of hair strands at a first location on the user's head;

(b) placing the first tuft of hair into a position extending through a first one of two slits that are found in an elongated flexible member of the head-worn apparatus proximate respective first end and second ends of said elongated flexible member so that the first tuft of hair passes from a first side of said elongated flexible member to a second side thereof;

(c) with the first plurality of hair strands secured together in the first tuft, and with the elongated flexible member spanning from the first location on the user's head to a different second location thereon along a hair part that likewise spans between the first and second locations on the user's head, taking a second tuft of hair formed from a second plurality of hair strands at said second location on the user's head and placing the second tuft of hair into a position extending through a second one of the two slits in the elongated flexible member from the first side of said elongated flexible member to the second side thereof; and

(d) maintaining the first and second pluralities of hair strands in said first and second tufts, thereby securing the head-worn appliance to the user's head.

According to a second aspect of the invention there is provided a method of applying a head-worn apparatus, the method comprising:

(a) forming a first tuft of hair on a user's head from a first plurality of hair strands at a first location on a user's head;

(b) forming a second tuft of hair on the user's head from a second plurality of hair strands at a second location on the user's head;

(c) creating a hair part that spans from proximate the first location to proximate the second location;

(d) engaging the first tuft of hair with a first slit in an elongated flexible member of the head-worn apparatus at the first location;

(e) laying the elongated flexible member along the hair part from where the first tuft of hair engages with the first slit in the elongated flexible member toward the second location on the user's head;

(f) engaging the second tuft of hair with a second slit in the elongated flexible member, thereby placing the apparatus in a position spanning between the first and second locations and securing the head-worn apparatus to the user's head.

Preferably step (f) comprises using a respective securing element that maintains the hair strands of the tuft together to also block sliding from a respective slitted area of the elongated flexible member toward a free end of said tuft.

The method may include engaging a respective securing element to each tuft of hair prior to placement thereof into the position passing through the respective slit in the elongated flexible member, whereupon the respective securing element maintains the hair strands within the tuft, whereby the securing elements secure the elongated flexible member in the position spanning between the first and second locations on the users head.

After engaging the second tuft with the second slit, the method may include adjusting a position of at least one of the securing elements to a point nearer to where the respective tuft emerges from the user's head in order to tighten the elongated flexible member between the first and second tufts.

At least one of the first and second securing elements may comprise a piece of tape.

At least one of the first and second securing elements may comprise a clip.

Preferably the elongated flexible member has a coarse texture, at least at areas thereof adjacent the first and second slits therein.

Preferably the elongated flexible member comprises a single unitary piece of flexible material spanning between the first and second ends.

The method preferably includes feeding additional strands of hair, from areas adjacent the hair part, through a series of holes found in the elongated flexible member at spaced apart positions there along between the first and second slits.

Preferably the first and second slits are free of any intersection with any perimeter edge of the elongated flexible member.

In one embodiment, the first and second locations are situated proximately rearward of a forehead hairline of the user's head on respective opposing first and second sides thereof, and step (d) comprises achieving and maintaining a tensioned state of the elongated flexible member between said first and second tufts, whereby the tensioned state of the elongated flexible member between said first and second tufts exerts a pulling force on said tufts in order to tighten facial areas located proximate to the first and second tufts.

In an alternative embodiment, the head-worn apparatus comprises an adornment carried on the elongated flexible member for display of said adornment on the user's hair.

Preferably the adornment is hung from the elongated flexible member via one or more holes provided in the elongated flexible member at an intermediate area between the first and second slits.

Preferably the head-worn apparatus further comprises one or more supports respectively engaged through the one or more holes in the elongated flexible member, and the adornment is hung from the head-worn apparatus via the one or more supports.

Preferably the one or more supports each comprise a U-shaped or V-shaped body and a pair of lateral arms projecting respectively outward from opposing sides of the body at an upper end thereof to hook the support to the elongated flexible member upon insertion of a lower end of the body through a respective one of the one or more holes in the elongated flexible member.

The adornment may comprise a jewelry chain coupled to the elongated flexible member.

The adornment may further comprise a jewelry ornament coupled to the jewelry chain, whereby the jewelry ornament is suspended from the elongated flexible member by the jewelry chain.

Opposing ends of the jewelry chain may be coupled to the elongated flexible member at two different points that are spaced apart along the elongated flexible member at an intermediate area between the first and second slits, and the jewelry ornament accordingly coupled to the jewelry chain at an intermediate portion thereof between the opposing ends of said jewelry chain.

According to a third aspect of the invention, there is provided a head-worn apparatus comprising an elongated flexible member having first and second slits therein proximate opposing first and second ends thereof for respective engagement of first and second tufts of hair through said first and second slits at respective first and second locations on the user's head to secure the head-worn apparatus in a position spanning between the first and second locations.

According to another aspect of the invention, there is provided a temporary facial tightening apparatus comprising an elongated flexible member having first and second slits therein proximate opposing first and second ends thereof for respective engagement of first and second tufts of hair through said first and second slits at respective first and second locations proximately rearward of a forehead hairline of the user's head on respective sides thereof to form a tensioned connection of said elongated flexible member between said first and second tufts and thereby tighten facial areas proximate the first and second locations of the first and second tufts.

According to yet another aspect of the invention there is provided a temporary facial tightening method comprising:

forming a first tuft of hair on a first side of a user's head from a first plurality of hair strands at a first location proximately rearward of a forehead hairline of the user's head;

placing the first tuft of hair into a position extending through a first one of two slits that are found in an elongated flexible member proximate respective first end and second ends of said elongated flexible member so that the first tuft of hair passes from a first side of said elongated flexible member to a second side thereof;

with the first plurality of hair strands secured together in the first tuft by a first securing element engaged to said tuft on the second side of said elongated flexible member, and with the elongated flexible member spanning from the first side of the user's head to an opposing second side thereof along a hair part that likewise spans between the first and second sides of the user's head, taking a second tuft of hair formed from a second plurality of hair strands on said second side of the user's head at a second location proximately rearward of the forehead hairline and placing the second tuft of hair into a position extending through a second one of the two slits in the elongated flexible member from the first side of said elongated flexible member to the second side thereof; and

using first and second securing elements to both maintain the first and second pluralities of hair strands in said first and second tufts to achieve and maintain a tensioned state of the elongated flexible member between said first and second tufts, whereby the tensioned state of the elongated flexible member between said first and second tufts exerts a pulling force on said tufts in order to tighten facial areas located proximate to the first and second tufts.

According to a further aspect of the invention there is provided a temporary facial tightening method comprising:

forming a first tuft of hair on a first side of a user's head from a first plurality of hair strands at a first location proximately rearward of a forehead hairline on the user's head;

forming a second tuft of hair on a second side of a user's head from a second plurality of hair strands at a second location proximately rearward of the forehead hairline;

creating a hair part that spans from proximate the first location to proximate the second location;

engaging the first tuft of hair with a first slit in an elongated flexible member at the first side of the user's head;

laying the elongated flexible member along the hair part from where the first tuft of hair engages with the first slit in the elongated flexible member toward the second location at the second side of the user's head;

engaging the second tuft of hair with a second slit in the elongated flexible member in a manner achieving a tensioned state of the elongated flexible member between the first and second tufts of hair, thereby tightening facial areas proximate the first and second locations of the first and second tufts.

### BRIEF DESCRIPTION OF THE DRAWINGS

In the accompanying drawings, which illustrate one or more exemplary embodiments of the present invention:

FIG. 1A is a plan view of temporary facial tightening kit according to one embodiment of the present invention, which is made up of a single elongated strip of flexible fabric and a pair of hair clips.

FIG. 1B is a plan view of temporary facial tightening kit according to another embodiment of the present invention, which is made up of a single elongated strip of flexible fabric and two pieces of adhesive tape.

FIG. 1C is a plan view of a jewelry kit according to another embodiment of the present invention, which uses the same elongated strip of flexible fabric together with a jewelry chain and associated item of jewelry to be hung in the user's hair using the fabric strip.

FIG. 2 illustrates initial steps of applying the facial tightening kit of FIG. 1A or 1B, in which strands of hair proximate each temple of the user's head are grouped into a respective tuft and maintained in such state by application of a respective one of the clips or tape pieces, and a hair part is created across the head behind the tufts.

FIG. 3 illustrates subsequent steps of applying the facial tightening kit, including passage of one hair tuft through a respective slit near one end of the fabric strip, and laying of the strip along the part in order to likewise receive the second tuft in the second slit and accept passage of additional hair strands through a series of intermediate openings in the strip between the two slits.

FIG. 4 illustrates the completed facial tightening application, in which the fabric strip is concealed beneath the hair that was previously raised to create the part along which the strip was laid, and the strip is maintained in a tensioned state between the tufts by the clips or tape that prevent the tuft from sliding out of the slits in the fabric strip.

FIG. 5 illustrates application of the jewelry kit of FIG. 1C using the same steps briefly described above in relation to FIG. 3.

FIG. 6 illustrates the completed jewelry application, in which the fabric strip is again concealed beneath the user's hair, while the chain and item and jewelry hang externally over the hair for ornamental display.

### DETAILED DESCRIPTION

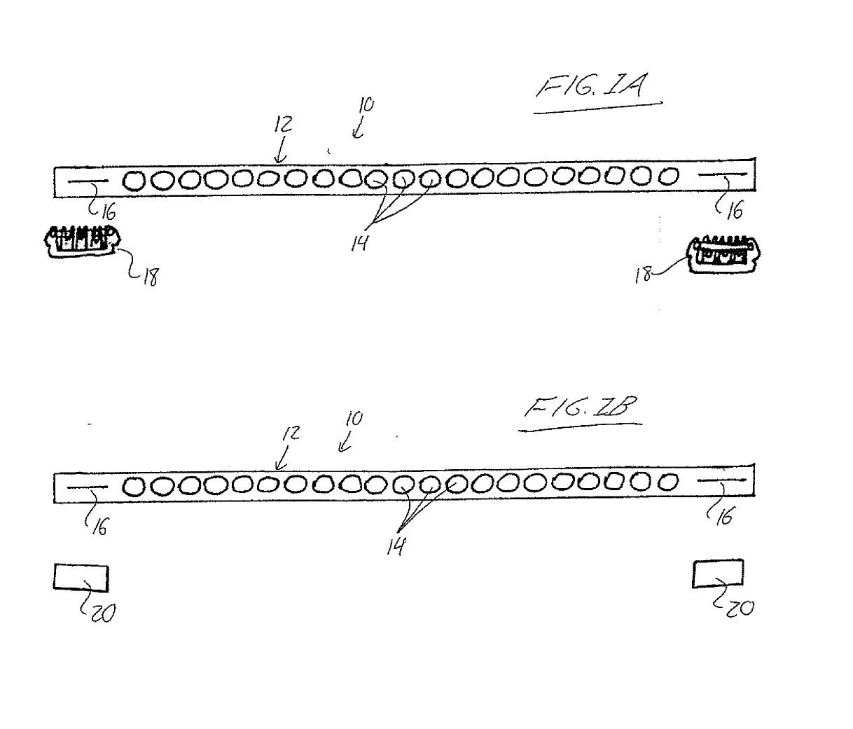

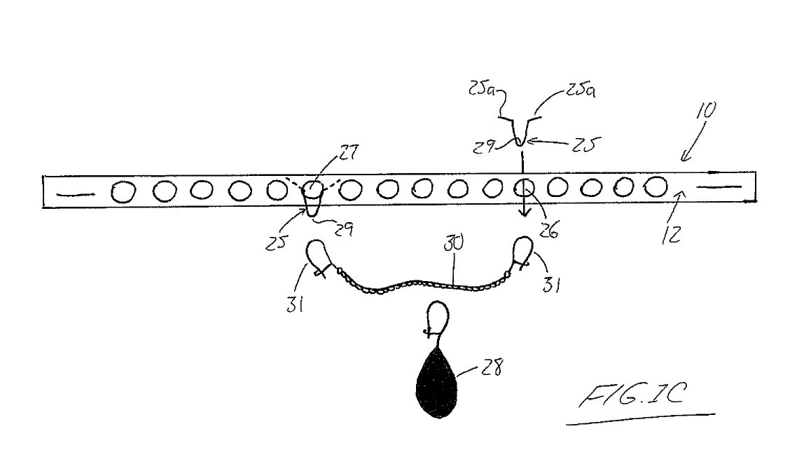

FIGS. 1A and 1B illustrate a hair appliance product 10 that is used to temporarily tighten the facial skin to improve the quality and appearance of the skin, removing/reducing wrinkles around the eyes and upper cheeks, and possibly also lower facial areas.

The appliance 10 of the illustrated embodiment is a single strip 12 of coarse, strong fabric, such as a narrow-width belting, which is a coarse fabric used to reinforce beltlines in garments. The fabric has a longitudinal dimension that greatly exceeds its width, and features a series of small holes 14 spaced apart along this longitudinal dimension, which may be formed by a hole punch. As described in further detail below, the strip 12 will be placed in the hair, at which point the holes or perforations 14 allow hair on the top of the user's head to be pulled through them, which holds the appliance in place in the worn position on the user's head and also partially conceals it from sight. Near both ends of the strip, a respective slit 16 is cut into the fabric. As shown, each slit may extend in the longitudinal direction of the strip, whereby the slit can have a length that exceeds the narrow width dimension of the strip without intersecting with any perimeter edge of the strip.

In one embodiment, the strip is approximately 9.5-inches in length. The strip may be painted (e.g. spray painted), died, or otherwise coloured on at least one side with the intention of matching or approximating the hair colour of a user. Accordingly, differently coloured strips may be offered by the manufacturer accordingly to various typical hair colours.

In the embodiment of FIG. 1A, the strip 12 is accompanied by a pair of hair extension snap-clips 18 of a known commercially available type, which features a generally rectangular open metal frame. One side of the frame carries a comb-like configuration of metal teeth or fingers that reach over the open center of the rectangular frame and then pass over a narrow resilient plastic or rubber band/strip on the other side of the clip's open rectangular frame. The side of the metal frame that carries the comb is bendable or flexible back and forth between two stable positions, particularly a closed positioning abutting the comb teeth or fingers against the resilient strip at the other side of the frame and an open position spacing the comb teeth or fingers away from the resilient strip.

As described below, each clip 18 is used to hold together a tuft of hair that is formed by grouping together a plurality hair strands, and also to help retain each such tuft in a position engaged with the strip 12 through one of the slits 16 therein. A third clip 18 can also be placed along the edge of the strip 12 to further ensure that the strip does not move when being worn.

In the second embodiment shown in FIG. 1B, the two clips 18 of the first embodiment are replaced with two pieces of adhesive tape 20, which are used for the same purpose of securing the hair strands of each tuft together, and helping prevent each tuft from sliding off its engaged position passing through the respective slit 16 in the appliance 10.

The strip 12 and clips 18 of the first embodiment, may be sold together in a kit, for example with similar colouring of these components, in order to best conceal all components of the kit within the user's hair when in use. Alternatively, the strip may be sold alone for use with separately purchased clips, which may or may not be manufactured and/or marketed by the same entity. Likewise, the tape 20 in the second embodiment may be provided as part of a ready-to-use kit, or the strip 12 may be sold alone on the expectation that the users already possess, or can readily obtain, a roll of adhesive tape from which they can cut appropriately sized pieces for taping up the tufts of hair.

Having described the physical apparatus, attention is now turned to how the appliance is applied. The appliance may be self-applied by the user, or applied to the user by another person.

Turning to FIG. 2, a group of individual strands of hair (for example, approximately 30 to 50 strands) are chosen and gathered together from in front of the ear, at a location near to but behind the forehead hairline of the user, thereby forming a tuft or cluster 22 of hair strands on one side of the user's head. One of the small clips 18 is engaged to this tuft of hair 22, or one the pieces of tape 20 is wrapped around this tuft of hair 22 at a location spaced from the rooted end of the tuft where the tufted hair strands emerge from the scalp of the user's head, preferably by between one and two inches.

The clip 18 may be of the aforementioned type currently available on the market for effectively holding hair extensions firmly in place. In such instance, to secure it in place, the clip 18 is opened and the tuft of hair is threaded or fed between the comb-side of the clip and the plastic strip side of the clip. The metal comb-carrying side of the clip is then bent away from the plane of the plastic strip into the closed position so as to tighten the plastic strip against the comb side of the clip, thus holding tufted-together strands of hair firmly together. Although an “extension clip” of the type referenced above has been found to be effective, other types of clips capable of holding the tuft together may alternatively be employed. If tape 20 is used instead, the tape is simply wrapped tightly around the tuft in order to cluster the hair strands tightly together in a secured bundle at the taped area.

As shown in FIG. 2, the clip or tape 18, 20 secures together a tuft of hair on one side of the head near the temple. As described herein further below, the secured-together tuft of hair will later be positioned behind the hairline where the tuft of hair and the clip or tape holding it together will not be seen.

The forgoing process of grouping together strands of hair into a tuft and securing the tufted hairs together is repeated at a generally matching location on the opposing side of the users head.

With continued reference to FIG. 2, the hair is parted so that the “part” 24 runs from the front of one ear over the top or upper back area of the head, and around to a generally matching location just in front of the other ear. The hair part 24 thus spans from a position adjacent the tuft on one side of the head to a position adjacent the other tuft on the opposing side of the head.

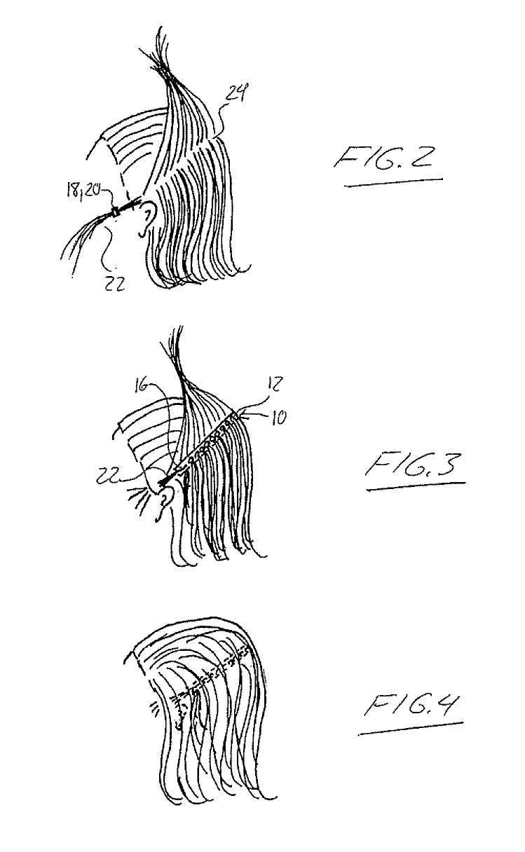

Turning to FIG. 3, the free end and the clipped or taped section of one of the tufts 22 is then passed through the slit 16 located near a respective end of the strip-shaped appliance 10. This tuft 22 will remain in place in the appliance 10 because the slit 16 is narrow and the fabric of the appliance is coarse and remains structurally strong so as to hold the clip or taped hair in place even when the tuft of hair is under pressure.

With one side of the apparatus in place as a result of this engagement of the first tuft with the first slit in the strip-shaped appliance 10, the appliance 10 is laid along the “part” 24 spanning over the head and is fastened in a similar manner to the second tuft of hair on the other side of the head. If only one surface of the strip-shaped appliance 10 features a suitable hair-approximating colour that blends with the user's hair, then the appliance is laid out in an orientation that faces this hair-coloured surface of the strip outwardly away from the underlying head of the user.

FIG. 3 shows the tuft 22 in the engaged position passing through the slit 16 of the strip-shaped appliance 10 from the outer-side or topside of the strip 12 that faces away from the user's head to the bottom-side or underside of the strip 12 that faces toward the user's underlying head. As a result, the clip 18 or tape 20 is concealed behind or beneath the strip 12 in FIG. 3. However, the tuft 22 may alternatively pass through the slitted area of the strip 12 in the opposite direction in its travel from the rooted end of the tuft to the opposing free end thereof. Sliding of the slitted area of the strip toward the free end of the tuft is blocked by the clip 18 or tape 20 that defines an enlargement on the tuft that doesn't fit through the naturally-closed default state of the slit 16, thus aiding the frictional grip of the course strip material on the tufted hair at the respective slit 16 in order to maintain the tuft and strip in secured positions relative to one another.

The clip/tape 18, 20 around each tuft of hair is placed at a location that will ensure that tension is put on the appliance when both tufts of hair are attached to the appliance, whereby the pulling force exerted on the tufts by the tensioned strip pulls on the scalp at the rooted ends of the tufts, thereby tightening the skin near the user's eyes and upper cheeks, and thus removing/reducing small lines and wrinkles at these facial areas when the appliance is in place. The strip material used may have very little, or no, resilient stretchability in its longitudinal direction. After the first end is engaged with the respective tuft, the strip is laid across the part 24, and is placed in a tensioned state by pulling the second tuft of hair far enough to be placed into slit 16 at the second end of the strip. The tension can be increased or decreased by moving the location of the clip/tape up or down on one or both tufts of hair. That is, if the clip or tape is moved ‘up’ the hair (i.e. toward the scalp at the rooted end thereof), the tension of the strip is increased in order to increase the tightening effect on the facial skin. If the clip or tape is moved ‘down’ the hair (i.e. away from the scalp and toward the free end of the tuft), the tension of the strip is decreased in order to reduce the pulling force on the scalp and tightening-effect on the facial skin.

With continued reference to FIG. 3, to ensure that the appliance 10 does not move, additional strands of hair around the crown of the head (i.e. adjacent or near the part 24) are pulled through all of the perforated holes 14 in the strip 12. A third clip 18 can be placed along the edge of the strip 12 to further hold the strip in place.

Turning to FIG. 4, after the appliance is in place, the hair above the part 24 (for example having been previously held in place by a hair clip or the like) may be released, which alone, or together with further styling of the hair, acts to conceal the appliance 10 and accompanying clips/tape 18, 20 from sight.

Commercially, additional natural or synthetic hair could be sewn onto the appliance to assist in further concealing it, or perforated holes could be made in different sizes and layout patterns, which would not change the basic characteristics and outcome of the appliance. For example, smaller holes aligned in two parallel rows along the lengthwise direction of the strip, with the intention that greater number of holes may better conceal the strip when the hair is fed through them. The holes may be of any such, including round, oval, elliptical, square, rectangular, triangular, pentagonal, hexagonal, etc. or any irregular shape. The intended primary use of the appliance is the temporary improvement of the appearance of the skin, for example to be worn by women who want to look more youthful and brighter on special occasions. Hair dressers may typically be responsible for putting the appliance in place, although the wearer could also perform the application.

While the illustrated embodiment features a single unitary integral strip of uniform width and composition throughout its length from one end to the other, other variations of an elongated flexible member may be employed for the appliance. For example, although the use of a perforated central portion of the strip is preferable to allow the wearer's hair to be fed through the holes 14 to help conceal and maintain the position of the appliance, another embodiment may employ slitted pieces of material that are joined together by a thinner, unperforated central strip, band or strand that is integrally formed with or otherwise attached to the slitted pieces of material to which the hair tufts are engaged. A reduced width between the slitted end pieces would still provide reasonable concealment beneath the overlying hair, even without perforations through which additional hair strands are fed, but the use of a perforated central region through which strands of hair are threaded is preferable in order to better contribute to steady maintenance of the appliance's position while worn.

Use of a single integral structure and the use of a uniform-width strip shape also contribute to simplified manufacture of the appliance. The use of tuft engaging slits in the appliance allows the use of clips, tape or other tuft-securing means that are separate and distinct from the strip or elongated member, thereby further simplifying the structure and manufacture of the appliance by avoiding the need to attach pre-installed clips or other fastening to the strip, as is required in some prior art clip-on or adhesively applied.

While the detailed placement or application process described above includes creation of both tufts and securing of same with the clips or tape prior to creating the part and engaging the first tuft with the strip, it may be possible to alternatively form and secure the first tuft and create the part, and then engage the first tuft with the strip and pull the second end of the strip over the part to the second side of the head, where the second tuft is then formed, secured and passed through the second slit. However, forming and securing both tufts in advance frees up both hands for use in engaging each slit with the respective tuft, thereby simplifying the process. Likewise, although the detailed embodiment discloses attachment of the clip or tape prior to each tuft before feeding same through the respective slit, it may be possible to pass the free end of a manually gripped tuft through the slit and then attach the clip or tape to that tuft. However, manipulation of the tuft through the respective slit may be easier to perform with the clip or tape already attached.

The temporary facial tightening appliance described above can also be used as a base for placing jewelry in the hair as shown in FIG. 1C and FIGS. 5 and 6. Additional components used for this purpose include one or more small supports 25 that each feature a U or V shaped body with a short extension 25a at the top of each of the two upright arms of the U or V-shaped body, such that the extensions 25a extend laterally outward from these arms by approximately half a centimeter in a generally horizontal direction. The main body 25 and lateral extensions 25a of the jewelry support can be formed from wire, plastic or other stiff material. A simple way of forming the jewelry support uses a commercially available hair styling pin. Each arm of the pin is cut at a length of 2 cm above the bottom of the U or V. The top end of each arm of the pin is then bent downward into a generally horizontal position at approximately 1.5 cm above the bottom of the U or V. It will be understood that these dimensions are exemplary only, and are not intended to limit the scope of the claimed invention.

After the strip 12 of the apparatus is placed in the hair in the same manner described above for the facial tightening embodiments, the lower end of the U or V-shaped body of the jewelry support 25 is then placed into a hole 26 in the hair appliance product 10 at a location along the strip 12 where the wearer wishes to place a piece of jewelry in the hair. The jewelry support is inserted, with the bottom of the U or V being inserted first, from the underside of the strip 12, through the hole 26 and out the topside of the strip 12, such that the bottom of the U or V hangs below the hair appliance product and the two horizontal arms of the jewelry support are caught on either side of the hole, thus preventing the jewelry support 25 from falling through the hole 26, 27 and effectively hooking it onto the strip 12.

A conventional earring 28, charm, pendant or other jewelry item that has a suitable fastener attached to it, is then placed through the bottom of the jewelry support 25. The jewelry item can use any form of fastener to hold it in place on the jewelry support, as long as the stopper for the fastener is larger than the size of bottom of the jewelry support 29, or can otherwise be clipped in place so that it does not fall off the jewelry support. After the jewelry item 28 is fastened into the jewelry support 25, the hair is smoothed down around the hair appliance product 10 and the jewelry, at which point the jewelry sits on the top of the hair while the hair appliance product is camouflaged or concealed by the hair. When used for jewelry adornment purposes in this manner, the appliance product does not have to be used for temporary skin tightening purposes, and therefore may not require as much tension in its applied state, provided that the achieved tension is sufficient to prevent the strip 12 from sliding out of its intended position on the user's head under the weight of the jewelry suspended form the strip by the jewelry support 25. When using the hair appliance product 10 only as a jewelry support system, the length of the hair appliance product can be any length and can be placed at any location the user wishes. In other words, the strip 12 does not necessarily need to extend from one side of the wearer's head to the other. However, when strung across the wearer's head from one side to the other, the appliance may be used simultaneously for both jewelry adornment and facial tightening purposes.

Another option for placing jewelry in the hair uses a jewelry chain 30 to which earring fasteners 31, or other small fasteners, are attached at opposing ends of the chain, either before or after the separate jewelry item 28 is coupled to the chain at an intermediate location between its opposing ends. Two jewelry supports 25 are placed in two different holes 26, 27 in the hair appliance product at points on opposite sides of the location at which the user wishes the jewelry to be located. Once the jewelry supports 25 are in place, the fasteners 31 at the ends of the chain are placed through the bottoms 29 of the jewelry supports 25. If jewelry is not already attached to the chain, one or more earrings, pendants, charms or other jewelry items can be attached to the chain. The jewelry item can use any form of fastener as long as the stoppers are larger than the size of holes in the chain or the fastener can otherwise be closed around the chain such that the jewelry does not fall off the chain. The hair is then smoothed down around and under the chain 30 to conceal or camouflage the jewelry supports 25 and the hair appliance product 10. It is also appreciated that because of the course nature of the strip 12, jewelry or chains can be fastened directly to the strip 12 at any location without the use of supports 25.

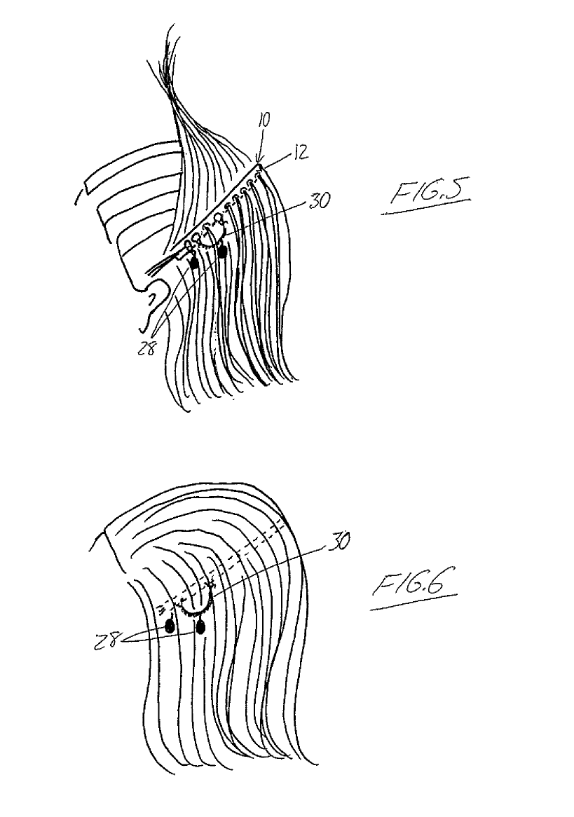

It will be appreciated that more than one chain can be placed on the hair appliance product in the same manner as outlined above. In addition, as an alternative to connecting both ends of the chain of the hair appliance 10 and coupling the jewelry item to the chain at an intermediate location between it ends, one could instead connect only one end of the chain to the hair appliance 10, and couple the jewelry item to the opposing end of the chain, which would hang freely from the appliance 10. FIGS. 5 and 6 illustrate how both chained and unchained jewelry items may be coupled to the same hair appliance 10 using different selected holes in the strip 12 of the appliance 10.

Since various modifications can be made in my invention as herein above described, it is intended that all matter contained in the accompanying specification shall be interpreted as illustrative only and not in a limiting sense.

1. A method of applying a head-worn apparatus, the method comprising:
(a) forming a first tuft of hair on a user's head from a first plurality of hair strands at a first location on the user's head;
(b) placing the first tuft of hair into a position extending through a first one of two slits that are found in an elongated flexible member of the head-worn apparatus proximate respective first end and second ends of said elongated flexible member so that the first tuft of hair passes from a first side of said elongated flexible member to a second side thereof;
(c) with the first plurality of hair strands secured together in the first tuft, and with the elongated flexible member spanning from the first location on the user's head to a different second location thereon along a hair part that likewise spans between the first and second locations on the user's head, taking a second tuft of hair formed from a second plurality of hair strands at said second location on the user's head and placing the second tuft of hair into a position extending through a second one of the two slits in the elongated flexible member from the first side of said elongated flexible member to the second side thereof; and
(d) maintaining the first and second pluralities of hair strands in said first and second tufts, thereby securing the head-worn appliance to the user's head.

2. A method of applying a head-worn apparatus, the method comprising:
(a) forming a first tuft of hair on a user's head from a first plurality of hair strands at a first location on a user's head;
(b) forming a second tuft of hair on the user's head from a second plurality of hair strands at a second location on the user's head;
(c) creating a hair part that spans from proximate the first location to proximate the second location;
(d) engaging the first tuft of hair with a first slit in an elongated flexible member of the head-worn apparatus at the first location;
(e) laying the elongated flexible member along the hair part from where the first tuft of hair engages with the first slit in the elongated flexible member toward the second location on the user's head;
(f) engaging the second tuft of hair with a second slit in the elongated flexible member, thereby placing the apparatus in a position spanning between the first and second locations and securing the head-worn apparatus to the user's head.

3. The method of claim 2 wherein step (f) comprises using a respective securing element that maintains the hair strands of the tuft together to also block sliding of a respective slitted area of the elongated flexible member toward a free end of said tuft.

4. The method of claim 1 comprising engaging a respective securing element to each tuft of hair prior to placement thereof into the position passing through the respective slit in the elongated flexible member, whereupon the respective securing element maintains the hair strands within the tuft, whereby the securing elements thereby secure the elongated flexible member in the position spanning between the first and second locations.

5. The method of claim 4 comprising, after engaging the second tuft with the second slit, adjusting a position of at least one of the securing elements to a point nearer to where the respective tuft emerges from the user's head in order to tighten the elongated flexible member between the first and second tufts.

6. The method of claim 1 wherein step (d) comprises using a respective piece of tape at one or both of the first and second tufts to maintain the respective hair strands therein.

7. The method of claim 1 wherein step (d) comprises using a respective clip at one or both of the first and second tufts to maintain the respective hair strands therein.

8. The method of claim 1 wherein the elongated flexible member has a coarse texture, at least at areas thereof adjacent the first and second slits therein.

9. The method of claim 1 wherein the elongated flexible member comprises a single unitary piece of flexible material spanning between the first and second ends.

10. The method of claim 1 comprising feeding additional strands of hair, from areas adjacent the hair part, through a series of holes found in the elongated flexible member at spaced apart positions therealong between the first and second slits.

11. The method of claim 1 wherein each of the first and second slits are free of any intersection with any perimeter edge of the elongated flexible member.

12. The method of claim 1 wherein the first and second locations are situated proximately rearward of a forehead hairline of the user's head on respective opposing first and second sides thereof, and step (d) comprises achieving and maintaining a tensioned state of the elongated flexible member between said first and second tufts, whereby the tensioned state of the elongated flexible member between said first and second tufts exerts a pulling force on said tufts in order to tighten facial areas located proximate to the first and second tufts.

13. The method of claim 1 wherein the head-worn apparatus comprises an adornment carried on the elongated flexible member for display of said adornment on the user's hair.

14. The method of claim 13 wherein the adornment is hung from the elongated flexible member via one or more holes provided in the elongated flexible member at an intermediate area between the first and second slits.

15. The method of claim 14 wherein the head-worn apparatus further comprises one or more supports respectively engaged through the one or more holes in the elongated flexible member, and the adornment is hung from the head-worn apparatus via the one or more supports.

16. The method of claim 15 wherein the one or more supports each comprise a U-shaped or V-shaped body and a pair of lateral arms projecting respectively outward from opposing sides of the body at an upper end thereof to hook the support to the elongated flexible member under insertion of a lower end of the body through a respective one of the one or more holes in the elongated flexible member.

17. The method of claim 13 wherein the adornment comprises a jewelry chain coupled to the elongated flexible member.

18. The method of claim 17 wherein the adornment further comprises a jewelry ornament coupled to the jewelry chain, whereby the jewelry ornament is suspended from the elongated flexible member by the jewelry chain.

19. The method of claim 17 wherein opposing ends of the jewelry chain are coupled to the elongated flexible member at two different points that are spaced apart along the elongated flexible member at an intermediate area between the first and second slits, and the jewelry ornament is coupled to the jewelry chain at an intermediate portion thereof between the opposing ends of said jewelry chain.

20. A head-worn apparatus comprising an elongated flexible member having first and second slits therein proximate opposing first and second ends thereof for respective engagement of first and second tufts of hair through said first and second slits at respective first and second locations on the user's head to secure the head-worn apparatus in a position spanning between the first and second locations.
